(19) United States
(12) Patent Application Publication
(10) Pub. No.: US 2012/0123324 A1
(43) Pub. Date: May 17, 2012

(54) DEVICE FOR INJECTING A SOLID

(75) Inventor: Christian SCHMALZ, Lyss (CH)

(73) Assignee: FORTEQ NIDAU AG, Nidau (CH)

(21) Appl. No.: 13/295,558

(22) Filed: Nov. 14, 2011

(30) Foreign Application Priority Data
Nov. 15, 2010 (EP) 10 405 220.4

(51) Int. Cl. A61M 5/31

(52) U.S. Cl. 604/63

(57) ABSTRACT

A syringe for injecting a solid medicament comprises a syringe body mounted movably in a housing and connected to a needle. A spring force acts on the syringe body in a proximal direction, wherein movement of the syringe body in the proximal direction can be freed by a first locking device that cooperates with the syringe body. A primary plunger, which is movable in the syringe body and in the needle, comprises a limit stop that can cooperate with the syringe body. The limit stop is spaced from the syringe body immediately prior to the unlocking of the first locking device. By way of the limit stop the primary plunger can be driven back in the proximal direction through the syringe body when the first locking device is unlocked. The first locking device can be deactivated by an actuating element arranged on the device.

### TECHNICAL FIELD

The invention relates to a device for injecting a solid into a human or animal body, said device comprising a syringe body mounted movably in a housing and connected to a needle, wherein a spring force acts on the syringe body in the proximal direction, and wherein the movement of the syringe body in the proximal direction can be freed by means of a first locking device that cooperates with the syringe body, and wherein the device further comprises a primary plunger, which is movable in the syringe body and in the needle, wherein the primary plunger comprises a limit stop that can cooperate with the syringe body, wherein the limit stop is spaced apart proximally from the syringe body in a state immediately prior to the unlocking of the first locking device, and wherein, by way of the limit stop of the primary plunger, the latter can be driven back in the proximal direction through the syringe body when the first locking device is unlocked.

### PRIOR ART

The injection of solids is a conventional procedure in long-term medication (depot injection). To this end, a depot is placed in the body and releases the medicament into the body over a predetermined period of time, ideally in a constant and continuous manner. Typical applications of long-term medication are in pain relief therapies, hormone therapies for contraception, etc. Injection of solids is also applied in conjunction with the subcutaneous injections of chips, for example microprocessors or storage chips.

A solid is typically injected by means of the solid being held by a plunger in a needle located in the body, whereupon the needle is drawn back, with the plunger remaining in place, and thus leaves the solid behind in the body.

WO 99/42148 A2 (LG Chemical LTD.; Feb. 20, 1998) discloses a syringe device for animals, which device comprises a protective sleeve and a housing. A needle, in which the material to be injected is held, is connected fixedly to a needle holder, which is mounted movably in the housing and on which a spring force acts in the proximal direction. The needle holder is held by a locking device in the distal end. The needle is guided into the body. The locking device is unlocked by pressing in the distal end of the housing, whereupon the spring relaxes and drives the syringe body with the needle back, in a first phase with the plunger remaining in place, and in a second phase together with the plunger.

NL 8901124 A (Nedap; May 3, 1989) discloses a device for injecting a solid, said device having a housing and an actuating element, which is connected fixedly to the plunger. Prior to the actuation, the hollow needle is located in the housing. When the actuating element is now moved down, the hollow needle with the plunger is guided into the body counter to a spring, which is tensioned in the process. After complete insertion, holding elements of the actuating element for holding the hollow needle are forced apart, whereupon the spring relaxes and the hollow needle is driven back into the housing, with the plunger remaining in place.

WO 99/42148 A2 and NL 8901124 A have the disadvantage that the solid is positioned at precisely a defined depth of penetration of the needle. Moreover, pressing the housing into the body must be considered unpleasant for the patient. If the deactivation does not take place directly upon insertion, the device has to be pressed with additional force onto the body, which can be unpleasant for the patient and can cause pain. However, if the locking device is set too sensitively, it can easily unlock too early if handled incorrectly. This can sometimes be very unpleasant if the needle is already inserted partially into the body and, as a result, the solid comes to lie only partially in the body. A further disadvantage is that the solid has to be held in the needle before the injection takes place. For this purpose, either the solid has to be subjected to a force that may damage the latter, or the possibility of the solid falling out of the needle prior to the injection has to be accepted.

### DISCLOSURE OF THE INVENTION

The object of the invention is to create a device for injecting solids that belongs to the technical field mentioned at the outset and that has a simple design and is safe to operate.

The object is achieved by the features defined in Claim 1. According to the invention, the first locking device can be deactivated by an actuating element arranged proximally on the device.

In the description below, “distal” designates a direction toward the body into which the solid is to be injected, and “proximal” designates the opposite direction, i.e. away from the body. In relation to the device, the two directions are each to be understood as being parallel to a longitudinal axis, wherein the longitudinal axis can be defined, for example, by the housing, the syringe body and the needle or by one of the two plungers. Unless otherwise stated, the directions “distal” and “proximal” are to be understood as being parallel to a longitudinal axis of the primary plunger.

The word “solid” is understood as the product that is to be introduced into the body. The solid can be designed as a solid medicament, as a container containing a liquid, gel or the like, or also as a microchip or the like. The device is not limited to a specific solid and instead can be used in a wide field, with the condition that the product to be injected, at least in an outermost layer thereof, is not liquid, in particular does not have too low viscosity. Preferably, the product to be injected is dimensionally stable in its outermost layer.

The device comprises a syringe body mounted movably in a housing and connected to a needle. Depending on the embodiment, the needle and the syringe body can also be designed in one piece. In particular, the syringe body can be designed as a section of the needle. For the needle, a protective cap can be provided that is removed before the device is used.

The syringe body is mounted movably in the housing typically by way of slide bearings, without movable parts. A person skilled in the art will be sufficiently aware of techniques for optimizing the guiding of the syringe body in the housing. These include, in particular, the choice of material of the individual parts, the choice of the contact surfaces, and the surface properties of the parts. Care must be taken in particular to ensure that the syringe body cannot become wedged in the housing. Of course, ball bearings or the like can also be provided in order to ensure satisfactory mounting of the syringe body, but the considerable outlay in design terms means that this option is best provided when the device is intended to be used more than once.

A spring force acts on the syringe body in the proximal direction. The spring force is preferably provided by a spring, in particular by a helical spring. The spring is preferably arranged in the distal direction with respect to the syringe body and is thus designed as a compression spring. In this way, a restoring force is easily made available for the syringe body. To be able to control the release of the spring, a locking device is provided, which is designed such that it can be deactivated (see below). A device for injecting a solid is thus created that is particularly ergonomic for the user, because the needle does not have to be driven back by hand. Such a movement can be tricky, since it typically takes place in the same direction in which the needle is pulled out of the body. In conventional injection devices in which the needle is drawn back by hand, particular care therefore has to be taken to ensure that the device itself is not drawn back, since otherwise the solid could be pulled out of the body.

In alternative embodiments, it is possible to use other elements that can exert a spring force. These include, for example, extension springs or rubber bands, which can act between the proximal end of the housing and the syringe body, but also fluid pressure systems with a compressible fluid, in particular a compressed air cartridge. Depending on the embodiment, however, such solutions may be technically more difficult to achieve.

The primary plunger is movable in the syringe body and in the needle and comprises a limit stop, which is spaced apart proximally from the syringe body prior to the unlocking of the first locking device. After the needle has been inserted, and the solid thus placed in the body, the locking device is unlocked in order, in a first phase, to draw the needle back with the primary plunger remaining in place. In this first phase, the syringe body reaches the limit stop of the primary plunger and, in a second phase, guides the latter back in the proximal direction. Immediately prior to the first phase, that is to say before the first locking device is unlocked, the primary plunger and the needle extend beyond the distal end of the housing, and the needle in turn extends beyond the distal end of the primary plunger. In this state, the solid is located in the interior of the needle, in front of the primary plunger. During the first phase, after the locking device has been unlocked, the needle is drawn back while the primary plunger remains in place, and the solid is maintained in position in the body by means of the primary plunger remaining in place. At the end of the first phase, the syringe body, which travels together with the needle, makes contact with the limit stop of the primary plunger. In this state, the primary plunger protrudes from the needle. It is unimportant here whether the needle lies completely in the syringe body or still protrudes therefrom. In the second phase, the primary plunger is moved together with the syringe body and the needle back in the proximal direction. At the end of the second phase, the needle and the primary plunger are preferably located inside the housing, thus providing a hygienic and safe end state of the device following the injection. By virtue of the needle and the primary plunger being located in the housing after the injection, it is possible to reduce the risk of the user being injured by the needle and also the risk of contamination.

The first locking device can be deactivated by an actuating element arranged proximally on the device.

By providing an actuating element for deactivation of the locking device, it is possible to ensure that the latter can be unlocked in a controlled manner and that the needle can thus also be driven back in a controlled way. This has in particular the advantage that the user is prepared for the release of the return mechanism and is not taken by surprise by it.

Moreover, the provision of an actuating element allows the user to place the solid at a variable depth under the skin. For this purpose, the user can insert the needle to the desired depth in the body, such that the solid comes to lie at the desired location, and can then actuate the actuating element. At this instant, the spring relaxes, as a result of which the syringe body is driven back together with the needle. The primary plunger remains in place in this phase, such that the solid can be maintained in position. After the needle has been drawn back in this first phase, the syringe body contacts the limit stop of the primary plunger and, in a second phase, moves back together with the latter until the needle and the primary plunger come to lie completely in the syringe body.

Arranging the actuating element at the proximal end of the device means that actuation can be performed in an ergonomic manner. A user is after all accustomed to actuating a conventional syringe at the proximal end, and this is reflected in an ergonomic manner by the arrangement of the actuating element of the device.

Prior to the use of the device, the solid is preferably held in the syringe body and can be transferred into the needle by means of the primary plunger. The solid can additionally be held in the syringe body by a retaining device, such that the solid cannot slip out of the needle prior to the injection. The retaining device can in principle be provided in a known manner. However, a retaining device is preferred that holds the solid back gently, that is to say a retaining device that applies no radial force or only a very slight radial force to the solid and instead holds the solid back simply by means of an element that protrudes distally into the syringe body in front of the solid. This can be achieved, for example, by means of axially oriented, elongate and resilient elements on the syringe body that have two radially inwardly protruding lugs, wherein the solid is held between the lugs. In a proximal area, the elements are held resiliently and pivotably on the syringe body. The deactivation of the retaining device is effected by means of the primary plunger which, upon insertion into the syringe body, contacts the proximal lugs and thus drives the elements radially outward and so releases the solid.

Alternatively, the solid can also be held in the needle. In this case, it may be advantageous, prior to the injection, for the solid to be retained in the needle by means of friction for example.

The locking device preferably comprises a resilient element and a rigid catch element cooperating with the latter, which elements are arranged on an outer face of the syringe body and on the inner face of the housing such that they engage each other in the locked state.

The resilient element can itself be made of a resilient material, for example a resilient plastic, metal or the like. Moreover, it can also comprise a rigid part, on which a spring force is exerted by means of a resilient part. For this purpose, a rigid plastic part, metal part or the like can be used on which a spring force is exerted by means of a spring, in particular a helical spring, leg spring, conical spring, etc., or by a resilient plastic part, for example a rubber band. The catch element can be designed as a protruding element or as a notch.

In principle, the locking can also be effected by other means known to a person skilled in the art. Instead of a resilient element, it is also possible to provide a merely pivotable or movable element, which is held indirectly by the spring, which applies a radial force to the syringe body, with a force fit, or by a clamping action of the spring. The locking device can also engage directly in the spring.

The resilient element is preferably designed as an elongate tongue, which is pivoted relative to a longitudinal axis in the locked state and which, by means of a radially applied force, can be pivoted substantially into the axial direction, as a result of which the locking device can be unlocked. The spring force is preferably made available by the resilient design of the tongue itself.

Alternatively, the resilient element can also be designed as a radially inwardly directed, spring-loaded pin, which protrudes into a notch of the syringe body. Other embodiments are also known to a person skilled in the art.

The resilient element is preferably arranged on the housing. A locking device of a simple design is thus achieved. The resilient element can be formed integrally on the housing or can be connected as a separate element to the housing.

Alternatively, the resilient element can also be connected to the syringe body.

The rigid catch element is preferably designed as a protruding lug, in particular formed integrally on the syringe body. For this purpose, the syringe body can be formed in one piece with the lug.

Instead of the lug, the proximal end of the syringe body can itself serve as a catch element, in which case the tongues cooperate with the end of the syringe body. Moreover, it is also possible to dispense with the rigid catch element, particularly if the tongue cooperates directly with the spring.

Several first locking devices arranged symmetrically with respect to a longitudinal axis are preferably provided, in particular two first locking devices lying opposite each other. It is possible in this way to avoid the syringe body becoming wedged in the housing, and a robust design of the return device of the needle is thus achieved.

However, if the syringe body is guided sufficiently in the housing, it is also possible to dispense with a locking device.

The first locking device can preferably be deactivated by means of an actuating element designed as a secondary plunger. A particularly user-friendly device is thus created, since it then requires only one actuating element for moving the solid into the needle and for unlocking the locking device. In particular, the construction of the device is also simplified in this way.

Alternatively, a separate actuating element can also be provided for the deactivation of the locking device. Such an actuating element can be arranged laterally on the housing for example, in which case the tongues are preferably connected to the syringe body and are pivoted inward by means of a radially oriented bolt, such that the spring can relax and drive the syringe body in the proximal direction.

The secondary plunger is preferably designed as a hollow cylinder, which is closed at the proximal end and is mounted movably in the housing. This ensures that, when the secondary plunger is driven in, the syringe body can be received in the secondary plunger. This permits a compact construction of the device, particularly in respect of a length of the device. The shape of the cross section of the secondary plunger preferably corresponds to that of the housing. The shape of the cross section is preferably circular but can also be square or oval or have some other shape. If the cross section is not circular, it is possible to ensure that the secondary plunger can be driven in the housing in a manner secure against twisting, which can add to the ergonomic aspect during use. The secondary plunger can also have axial apertures on the sides. Structures for increasing the stability can be provided on the inside. Other variants are also familiar to a person skilled in the art.

In alternative embodiments, the secondary plunger can also be provided as a rod-shaped element with a head for actuation.

The first locking device can preferably be unlocked when the secondary plunger, located in the state of insertion in the housing, makes contact with the resilient element. This provides particularly ergonomic unlocking of the locking device since, as with a conventional syringe, the user has to drive the plunger into the housing for this purpose. When the secondary plunger is driven in, its distal end comes into contact with the tongue of the locking device and pivots this into the axial direction, as a result of which the tongue is no longer in engagement with the rigid catch element. In this way, the syringe body is no longer held back and can be driven in the proximal direction by the spring force.

As has been mentioned above, there are also other possible ways of unlocking the locking device.

The secondary plunger preferably has, at a distal end, a taper increasing continuously in the distal direction such that, in the inserted state of the secondary plunger, the resilient element is guided radially outward. This has the effect that the secondary plunger can grip the distally protruding tongues without being blocked by them before they are guided outward. Moreover, the secondary plunger can be designed such that it is guided snugly in the housing, that is to say is substantially free of play. Depending on the embodiment, and particularly if the secondary plunger is mounted secure against twisting in the housing, the taper can also be provided only locally at those sites where contact with the tongues can occur. In the case of a secondary plunger secured against twisting, it is also possible merely to provide an indent extending as far as the distal end, provided that the return of the syringe body is not impeded by this.

Alternatively, it is also possible to do without the taper. In this case, it can be advantageous if, behind the tongues, a recess for receiving the tongue is provided in the housing.

The secondary plunger is preferably connected releasably to the primary plunger. The fact that the secondary plunger is connected to the primary plunger means that the primary plunger, which transfers the solid into the needle, can be actuated together with the secondary plunger, in particular by means of the secondary plunger. This simplifies the operation of the device. The primary plunger is preferably held releasably at a distance from the proximal end of the secondary plunger. This has the effect that, in the released state, the primary plunger is able to move in the proximal direction inside the secondary plunger. Thus, during the return movement of the syringe body, this permits the second phase in which the primary plunger moves together with the syringe body.

In alternative embodiments, the secondary plunger can also be connected fixedly to the primary plunger or can also be formed in one piece. In this case, however, in the second phase of the return movement of the needle with the secondary plunger, the primary plunger would also be moved in the proximal direction. In this phase, the user would have to take particular care not to impede the proximal movement of the secondary syringe body.

The secondary plunger preferably has, on its inside in the proximal area, resilient latching hooks, which are designed as a second locking device for locking the limit stop of the primary plunger. The primary plunger preferably has corresponding elements that can engage with the resilient latching hooks. A particularly simple second locking device is thereby obtained.

In alternative embodiments, the primary plunger can also be connected to the secondary plunger by means of a perforated connection or via a predetermined break point. Other possibilities by which the primary plunger can be held releasably on the secondary plunger are known to a person skilled in the art.

The second locking device can preferably be unlocked by the syringe body. During the return movement of the needle, the syringe body is the movable element nearest to the locking device. Thus, a particularly simple unlocking of the second locking device is achieved. To permit unlocking, the syringe body contacts and actuates the resilient latching hooks at the end of the first phase and thus releases the second locking device, whereupon the syringe body can move in the proximal direction together with the primary plunger.

In alternative embodiments, the second locking device can also be unlocked manually using a separate actuating element.

The limit stop is preferably designed as an element protruding radially from the primary plunger, in particular as a radially oriented circular disk. The fact that the protruding element is rotationally symmetrical ensures, particularly in the case of a secondary plunger not secured against twisting, that the latching hooks come into engagement with the protruding element even in the event of twisting of the secondary plunger. It is thus possible to avoid the primary plunger having to be turned too and possibly damaged. The resilient latching hooks are preferably designed as axially oriented and radially pivotable tongues, which on the inner side have recesses for receiving the limit stop. In this way, the circular disk-shaped area can be received in a simple manner. For this purpose, the tongues are connected at the proximal end to the base area and are connected laterally, in the proximal area, to the inside wall of the closed secondary plunger or formed integrally thereon. The radially pivotable tongues can preferably be driven radially outward by the syringe body, as a result of which the second locking device can be released. For this purpose, in a distal area, the tongues are spaced apart slightly from the inside wall of the secondary plunger. Preferably, at least two opposite tongues are provided, particularly preferably three uniformly arranged tongues. However, it is also possible to provide more than three tongues or, if the primary plunger is guided in a sufficiently stable manner, precisely one tongue.

The limit stop can in principle be of any desired shape. For example, it can be advantageous, when using more than two tongues, if the limit stop is designed as a corresponding n-gou, in which case each tongue engages one side. The straight area of the limit stop can then ensure a secure engagement. Other shapes, for example an oval shape or a suitable polygonal shape, are also not excluded.

The housing preferably comprises, in a proximal area, a circumferential flange or two opposite, radially outwardly protruding grip elements. This allows the device to be operated analogously to a conventional syringe. This is advantageous especially if, in accordance with a preferred embodiment, the secondary plunger represents the single actuating element. During use, the user can place index finger and middle finger behind the two flanges and drive the secondary plunger in with the thumb. In a preferred variant, the proximal end of the secondary plunger also has a radially outwardly protruding projection, as a result of which a securing element can be fitted between the projection and the flanges. A releasable fixing can be obtained for example by a clip attachment on the secondary plunger. The securing element ensures that the secondary plunger is not accidentally driven into the housing before use. This securing element is removed before use.

It will be clear to a person skilled in the art that the flanges can in principle be of a different design. In particular, exactly one circumferential flange could also be provided.

Further advantageous embodiments and combinations of features of the invention will become clear from the following detailed description and from the complete set of claims.

### BRIEF DESCRIPTION OF THE DRAWINGS

In the drawings used to explain the illustrative embodiment and depicting a schematic cross section through the device:

FIG. 1 shows the device with the needle inserted into the body, before the secondary plunger is driven in;

FIG. 2 shows the device after the secondary plunger has been driven in, with the first locking device deactivated, and before the syringe body is driven back;

FIG. 3 shows the device with the syringe body driven back and with the second locking device deactivated, before the primary plunger is driven back;

FIG. 4 shows the device in the end state.

Identical parts are in principle provided with identical reference signs in the figures.

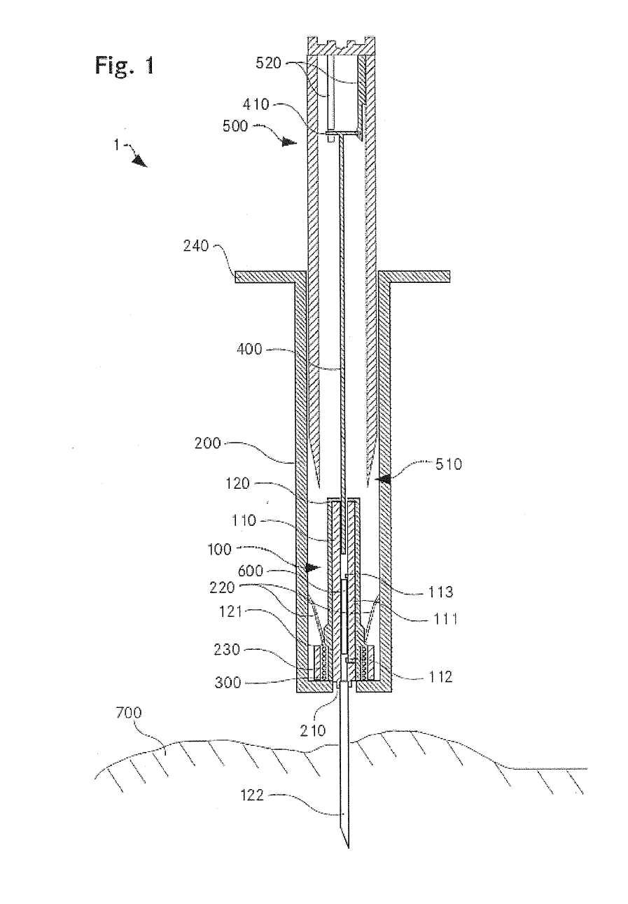

FIG. 1 shows an embodiment of a device 1 according to the invention for injecting a solid 600 into a body 700.

The device 1 comprises a housing 200, which is designed substantially as a circular cylinder, closed at one end, with a central opening 210 in the base and with a radially outwardly protruding flange 240 as grip element at the proximal end. The housing 200 further comprises an axially oriented guide sleeve 230, which is arranged on the base and has an internal diameter greater than the opening 210 and in which a helical spring 300 is guided coaxially, which is compressed in FIG. 1. The housing 200 further comprises, on the inner wall in a distal area, two opposite tongues 220, which are resilient and, in the rest state, are pivoted inward in the distal direction.

The device 1 further comprises a syringe body 100 composed of two parts, namely of a syringe body sleeve 120 and of a retaining device 110 arranged in the syringe body sleeve 120. The retaining device 110 is connected to a needle 122, wherein the through-opening of the retaining device communicates with a passage in the needle 122.

The retaining device 110 is arranged and secured inside the syringe body sleeve 120. For this purpose, the syringe body sleeve 120 comprises, at the proximal end, a radially inwardly protruding flange, which serves as a limit stop for the retaining device 110. The syringe body sleeve 120 has a substantially rotationally symmetrical design and comprises, on the outside, a radially outwardly protruding catch element 121, which is designed as a circumferential flange. The distal face of the catch element 121 is in contact with the compressed helical spring 300, and the proximal face is in contact with the tongues 220. The tongues 220 thus form a first locking device. The retaining device 110 is designed substantially as a hollow cylinder with a through-opening and comprises an elongate element 111, which is designed as a U-shaped recess in the hollow cylinder. The elongate element 111 comprises radially inwardly protruding lugs 112, 113, of which one lug 112 is integrally formed distally, and another lug 113 proximally, on the elongate element 111. Prior to the injection, the solid 600 is held between the two lugs 112, 113. The elongate element 111 is resilient, wherein the lugs 112, 113, in a rest state, protrude into the through-opening. If the primary plunger 400 is now inserted into the retaining device 110, it first contacts the proximal lug 113 and drives this radially outward together with the elongate element 111, as a result of which the distal lug 112 frees the path for the solid 600 into the needle 122. In order to permit the pivoting of the elongate element 111 inside the syringe body sleeve 120, the former is spaced apart from the inside wall of the syringe body sleeve 120. This spacing is not shown but can be obtained in various ways. For example, the elongate element 111 can be trimmed slightly on the outside, such that its material thickness is less than that of the rest of the retaining device 110. However, it is also possible for the syringe body sleeve 120 to be trimmed slightly in this area.

The primary plunger 400 is designed as a thin, long, circular cylindrical rod and comprises, at the proximal end, a limit stop 410 designed as a radially oriented circular disk.

The device 1 further comprises a secondary plunger 500, which is designed substantially as a circular cylinder closed at one end. In the direction of the opening, that is to say in the distal direction, the secondary plunger 500 has a continuous outside taper 510 which, when the secondary plunger 500 is inserted into the housing 200, can contact the tongues 220 and force them radially outward and unlock the first locking device. On the inside, the secondary plunger 500 comprises three tongues 520, of which only two are shown in each of the figures, of which one lies in the cross-sectional plane of the drawing and is therefore hatched. The tongues 520 are substantially rod-shaped and are connected in a proximal area to the inside wall, and at the proximal end to the base area, of the secondary plunger 500. In a distal area, the tongues 520 have less radial thickness than in the proximal area, as a result of which a spacing from the inside wall of the secondary plunger 500 is obtained. In this way, the distal area of the tongues 520 can be pivoted radially outward in a resilient manner. On the inner side, in the distal area, the tongues 520 have a notch in which the limit stop 410 of the primary plunger 400 is latched, as a result of which the second locking device is created. At the distal end, the tongues 520 have, on the inner side adjacent to the notches, outwardly directed bevels oriented in the distal direction. These bevels serve as a contact area for the syringe body 100, as a result of which the latter can drive the tongues 520 radially outward and can thus deactivate the second locking device.

FIG. 1 finally shows a schematic representation of a body 700 into which the solid 700 is to be injected.

FIG. 1 shows the device 1 in the state prior to the injection, with the needle already inserted into the body 700. The solid 600 is held between the lugs 112, 113 of the retaining device 110. The primary plunger 400 is latched by way of the limit stop 410 in the tongues 520 and is arranged with the distal end inside the retaining device 110 of the syringe body, before the proximal lug.

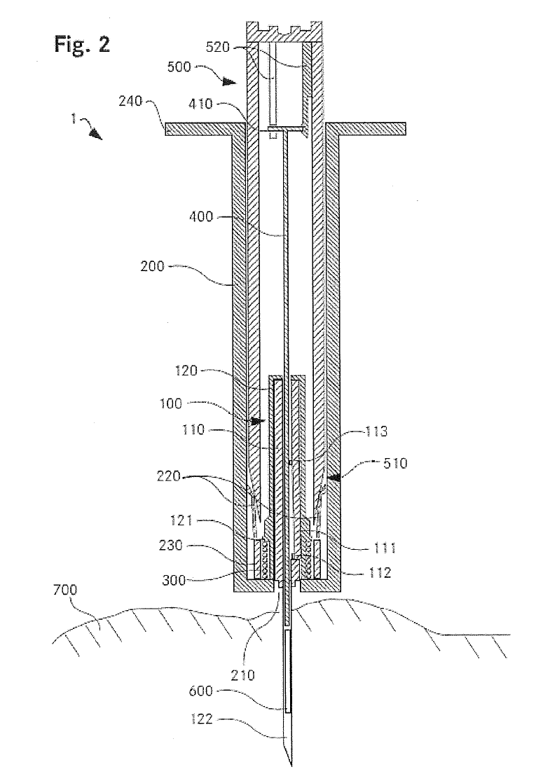

FIG. 2 shows the device 1 substantially as per FIG. 1, with the secondary plunger 500 fully inserted. At the same time, the primary plunger 400 is also fully inserted in this way and has already transferred the solid 600 into the needle 122 inside the body 700. The elongate element 111 of the retaining device 110 is pivoted radially outward. The tongues 220 are pivoted radially outward across the area of the taper 510 of the secondary plunger 500 and are thus no longer in engagement with the catch element 121 of the syringe body sleeve 120, as a result of which the first locking device is unlocked. The syringe body 100 is therefore no longer held back and can be driven in the proximal direction by means of the helical spring 300.

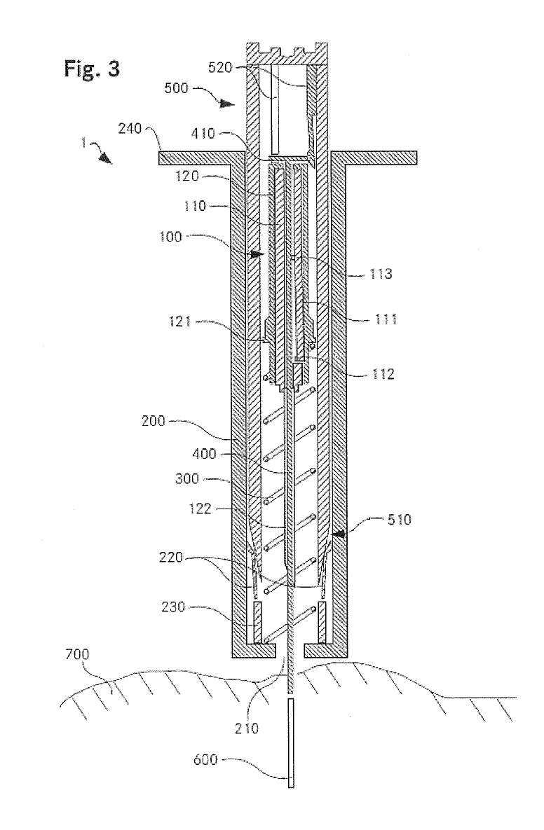

FIG. 3 shows the device 1 substantially as per FIG. 2, with the helical spring 300 now partially relaxed. The syringe body 100 is driven in the proximal direction together with the needle 122, with the primary plunger 400 remaining in place, and its proximal end contacts the bevels of the tongues 520 and drives the latter radially outward. The limit stop 410 is thus released from the tongues 520, whereupon the syringe body 100 can contact the limit stop 410 and, together with the primary plunger 400, can be driven farther in the proximal direction. The solid 600 is meanwhile positioned in the body 700, with the primary plunger 400 still extending into the body 700.

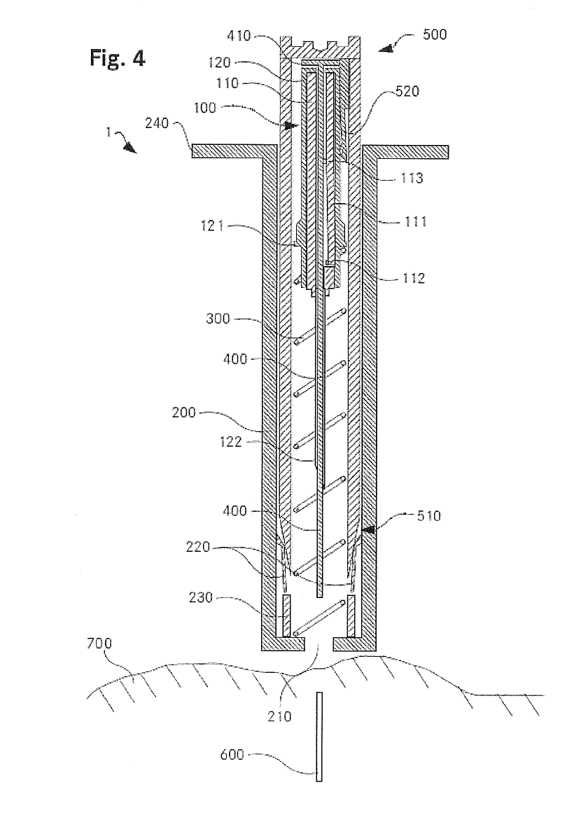

FIG. 4 shows the device 1 substantially as per FIG. 3 in its end state after the injection. The helical spring 300 has now guided the syringe body 100 and the needle 122, together with the primary plunger 400, as far as the limit stop of the secondary plunger 500, as a result of which the distal, contaminated areas of the needle 122 and of the primary plunger 400 now lie inside the housing 200.

In alternative embodiments, the housing 200 can also comprise the guide sleeve 230 and, in particular, can be formed in one piece with the guide sleeve. Correspondingly, the tongues 520 can also be formed in one piece with the secondary plunger 500.

It will be noted in summary that, according to the invention, a device 1 for injecting a solid 600 is created that has a particularly simple design and is also easy to use.

1. Device for injecting a solid into a human or animal body, said device (1) comprising:
a) a syringe body mounted movably in a housing and connected to a needle; wherein
b) a spring force acts on the syringe body in the proximal direction, wherein the movement of the syringe body in the proximal direction can be freed by means of a first locking device that cooperates with the syringe body;
c) a primary plunger, which is movable in the syringe body and in the needle; wherein
d) the primary plunger comprises a limit stop that can cooperate with the syringe body, wherein the limit stop is spaced apart proximally from the syringe body in a state immediately prior to the unlocking of the first locking device; and wherein
e) by way of the limit stop of the primary plunger, the latter can be driven back in the proximal direction through the syringe body when the first locking device is unlocked;
characterized in that
f) the first locking device can be deactivated by an actuating element arranged proximally on the device.

2. Device according to claim 1, characterized in that, prior to the use of the device the solid is held in the syringe body and can be transferred into the needle by means of the primary plunger.

3. Device according to claim 1, characterized in that the first locking device comprises a resilient element and a rigid catch element cooperating with the latter, which elements are arranged on an outer face of the syringe body and on the inner face of the housing such that they engage each other in the locked state.

4. Device according to claim 3, characterized in that the resilient element is designed as an elongate tongue, which is pivoted relative to a longitudinal axis in the locked state and which, by means of a radially applied force, can be pivoted substantially into the axial direction, as a result of which the first locking device is unlocked.

5. Device according to claim 3, characterized in that the resilient element is arranged on the housing.

6. Device according to claim 3, characterized in that the rigid catch element is designed as a protruding lug.

7. Device according to claim 1, characterized in that several first locking devices arranged symmetrically with respect to a longitudinal axis are provided, lying opposite each other.

8. Device according to one claim 1, characterized in that the first locking device can be deactivated by means of an actuating element designed as a secondary plunger.

9. Device according to claim 8, characterized in that the secondary plunger is designed as a hollow cylinder, which is closed at the proximal end and is mounted movably in the housing.

10. Device according to claim 8, characterized in that the first locking device can be unlocked when the secondary plunger, located in the state of insertion in the housing, makes contact with the resilient element.

11. Device according to claim 10, characterized in that the secondary plunger has, at a distal end, a taper increasing continuously in the distal direction such that, in the inserted state of the secondary plunger, the resilient element is guided radially outward.

12. Device according to claim 8, characterized in that the secondary plunger is connected releasably to the primary plunger.

13. Device according to claim 8, characterized in that the secondary plunger has, on its inside in the proximal area, resilient latching hooks, which are designed as a second locking device for locking the limit stop of the primary plunger.

14. Device according to claim 13, characterized in that the second locking device can be unlocked by the syringe body.

15. Device according to claim 13, characterized in that
a) the limit stop is designed as an element protruding radially from the primary plunger;
b) the resilient latching hooks are designed as axially oriented and radially pivotable tongues, which on the inner side have recesses for receiving the limit stop; wherein
c) the radially pivotable tongues can be driven radially outward by the syringe body, as a result of which the second locking device can be released.

16. Device according to claim 1, characterized in that the housing comprises, in a proximal area, a circumferential flange or two opposite, radially outwardly protruding grip elements.

17. Device according to claim 6, characterized in that the protruding lug is formed integrally on the syringe body.

18. Device according to claim 15, characterized in that the limit stop is designed as a radially oriented circular disk.

19. Device according to claim 3, characterized in that the first locking device can be unlocked when the secondary plunger, located in the state of insertion in the housing, makes contact with the resilient element.

20. Device according to claim 4, characterized in that the first locking device can be unlocked when the secondary plunger, located in the state of insertion in the housing, makes contact with the resilient element.
